(19) United States
(12) Patent Application Publication
(10) Pub. No.: US 2026/0028615 A1
(43) Pub. Date: Jan. 29, 2026

(54) EXTRACTION METHOD FOR NUCLEIC ACIDS

(71) Applicant: Siemens Healthcare Diagnostics Inc., Tarrytown, NY (US)

(72) Inventors: Ishita CHAKRABORTY, Bangalore (IN); Divya KHANDIGE, Bangalore (IN); Nivedita MITRA, Bangalore (IN); Ramya V M, Bangalore (IN)

(21) Appl. No.: 18/997,217

(22) PCT Filed: Jul. 26, 2023
(86) PCT No.: PCT/US2023/028644

§ 371 Date: Jan. 21, 2025

(30) Foreign Application Priority Data
Jul. 26, 2022 (IN) 202241042648

(51) Int. Cl. C12N 15/10; C07H 1/08

(52) U.S. Cl. CPC C12N 15/1013; C07H 1/08

(57) ABSTRACT

A method (100) of extracting nucleic acids is disclosed. The method includes receiving a sample containing nucleic acids to be extracted. The method (100) includes lysing cells in the sample to release the nucleic acids. The method (100) also includes introducing the released nucleic acids to a substrate, wherein the nucleic acids bind to the substrate. The method (100) includes washing the substrate bound to the nucleic acids and eluting the nucleic acids from the substrate. The introducing of the released nucleic acids to the substrate. the washing of the substrate bound to the nucleic acids, and the eluting of the nucleic acids are performed in a pipette tip.

### PRIORITY

This application claims the benefit of Indian Patent Application No. IN 202241042648, filed on Jul. 26, 2022, which is hereby incorporated by reference in its entirety.

### FIELD OF TECHNOLOGY

The present disclosure relates to the field of analysis of a sample and, more particularly, to the field of extraction of nucleic acids from a sample.

### BACKGROUND

Nucleic acid extraction is a critical upstream step in molecular diagnostic workflows. Current sample preparation methods involve long turnaround times and complex purification steps. This makes it complicated to integrate the sample preparation workflows with downstream steps. Rapid sample preparation methods often result in crude sample extract, which may not yield the required detection sensitivity. Additionally, many sample preparation methods may not yield maximum quantity of nucleic acids. There is also a need for the sample preparation methods to be adapted according to the sample type (e.g., blood, sputum, urine, etc.) and the target cells from which nucleic acids are to be extracted.

### SUMMARY

There exists a need for a method of nucleic acid extraction that is rapid, universal, automatable, and efficient and which when combined with a downstream nucleic acid amplification method, provides a sensitive diagnostic result in the shortest turnaround time.

A method of extracting nucleic acids from a sample is disclosed. In one aspect, the method includes receiving the sample including nucleic acids to be extracted. The method also includes lysing cells in the sample to release the nucleic acids. The method also includes introducing the released nucleic acids to a substrate, where the nucleic acids bind to the substrate. The method includes washing the substrate bound to the nucleic acids and eluting the nucleic acids from the substrate. The introducing of the released nucleic acids to the substrate, the washing of the substrate bound to the nucleic acids, and the eluting of the nucleic acids are performed in a pipette tip.

This summary is provided to introduce a selection of concepts in a simplified form that are further described below in the following description. The summary is not intended to identify features or essential features of the claimed subject matter. Further, the claimed subject matter is not limited to implementations that solve any or all disadvantages noted in any part of this disclosure.

### BRIEF DESCRIPTION OF THE DRAWINGS

The present invention is further described hereinafter with reference to illustrated embodiments shown in the accompanying drawings, in which:

FIG. 1 illustrates one embodiment of a method of extracting nucleic acids from a sample.

FIG. 2 illustrates one embodiment of a method of washing substrate bound to nucleic acids.

FIG. 3 illustrates one embodiment of a method of eluting nucleic acids from the substrate.

FIG. 4 illustrates a graphical representation-based comparison of binding efficiency of nucleic acids to the substrate by shaking and by using a pipette tip, according to an embodiment.

FIG. 5 illustrates a graphical representation of an effect of pH of elution buffer on the percentage of nucleic acids eluted, according to an embodiment.

### DETAILED DESCRIPTION

Hereinafter, embodiments for carrying out the present invention are described in detail. The various embodiments are described with reference to the drawings, where like reference numerals are used to refer to like elements throughout. In the following description, for purpose of explanation, numerous specific details are set forth in order to provide a thorough understanding of one or more embodiments. It may be evident that such embodiments may be practiced without these specific details. In other instances, well known materials or methods have not been described in detail in order to avoid unnecessarily obscuring embodiments of the present disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the present disclosure. Disclosed embodiments provide a method of extracting nucleic acids from a sample.

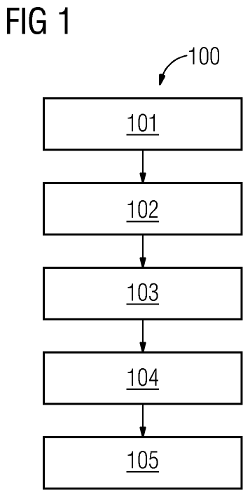

FIG. 1 illustrates one embodiment of a method 100 of extracting nucleic acids from a sample, according to an embodiment of the invention. The method includes a step 101 of receiving a sample including nucleic acids to be extracted. The sample may be, for example, blood, urine, sputum, cerebrospinal fluid, etc. The sample may include a plurality of cells from which nucleic acids are to be extracted. For example, the cells from which nucleic acids are to be extracted may include pathogens such as bacteria, virus, fungi, apart from the cells from human whole blood such as WBCs, RBCs, etc. At step 102, the cells from which the nucleic acids are to be extracted are lysed. The cells may be lysed, for example, using mechanical methods or chemical methods. For example, the mechanical methods include subjecting the sample to ultrasound-based lysis. In an embodiment, ultrasound lysis of the sample includes indirect ultrasound lysis. This includes subjecting the sample to ultrasonic waves at 60-100 KHz, for ten to fifty ‘one second to fifteen seconds ON and one second to fifteen seconds OFF’ cycles. Other mechanical methods of lysis include bead beating. In chemical-based lysis, the sample may be introduced to a chemical lysis buffer that includes a chaotrope, a salt, a detergent, and a buffering agent. The chaotrope may be in a concentration range of 1 to 10 M. The salt may be in a concentration range of 1 to 300 mM. Detergent may have a concentration of 1 to 15%. The lysis buffer may have a pH in the range of 6 to 8. In an embodiment, the lysis buffer may also include proteinase K, lysozyme, and/or other enzymes for lysing the cells. In another embodiment, the sample may be treated with proteinase K before introducing the lysis buffer. The mixture created between the sample and the chemical lysis buffer is heated to a temperature ranging between 60° C. to 90° C. This enables lysis of the cells in the sample. For example, viruses present in the sample may be lysed in 15 seconds to 4 minutes, using the chemical lysis buffer. On lysis of the cells, the nucleic acids from the cells are released into the mixture.

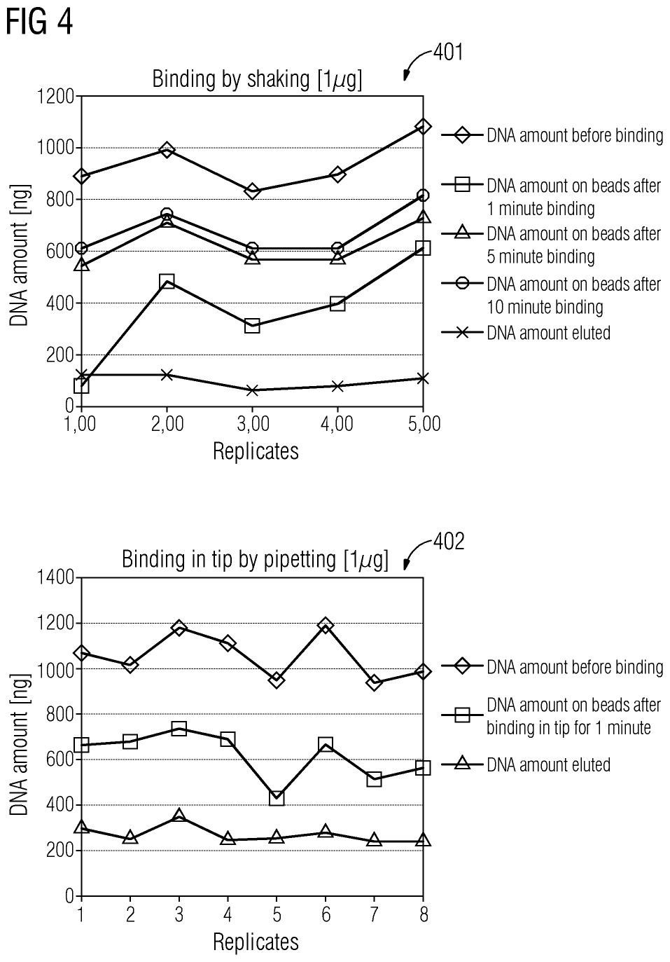

The method 100 further includes a step 103 of introducing the nucleic acids to a substrate such that the nucleic acids bind to the substrate. The nucleic acids from the lysed sample are introduced to the substrate in a pipette tip. The substrate may be, for example, silica coated paramagnetic beads. In an embodiment, the binding of the nucleic acids to the substrate is facilitated by aspiration and dispension of the lysed sample in the pipette tip. A non-orbital movement of the lysed sample across a length of the pipette tip enables effective binding of the nucleic acids to the substrate. Orbital movement-based methods of binding nucleic acids to the substrate, such as shaking the lysed sample and the substrate mixture in an orbital shaker, is less efficient in binding nucleic acids to the substrate in comparison with non-orbital movement-based method of binding. Non-orbital movement-based methods may also include vortexing, vibration, etc. (e.g., any rapid, non-laminar, turbulent liquid motion). In one embodiment, non-orbital movement of the lysed sample and substrate provides that the substrate is exposed to the entire volume of the lysed nucleic acid sample. The lysed sample is introduced to the substrate and subjected to heat at a temperature ranging between 45° C. and 75° C. A non-orbital movement of the mixture is performed in the pipette tip between 30 seconds to 5 minutes. FIG. 4 illustrates a graphical representation-based comparison of binding efficiency of nucleic acids to the substrate by shaking and by using a pipette tip, according to an embodiment. Referring to FIG. 4, the binding efficiencies of nucleic acids to the substrate using a non-orbital movement and orbital movement are measured using real-time PCR. As observed in graph 402, yield of nucleic acids in eluate is higher in the shortest binding time tested (e.g., when binding is performed by a non-orbital movement for one minute) compared to the yield of nucleic acids in eluate when binding is performed by an orbital movement for a binding time of one minute (e.g., in this case by shaking), as observed in graph 401. Prolonged shaking may also inhibit elution of nucleic acids, as indicated in the graph 401 and 402. The amount of nucleic acids eluted is higher when the nucleic acids are bound to the substrate in a non-orbital manner (e.g., pipetting) compared to when the nucleic acids are bound to the substrate in an orbital manner (e.g., shaking).

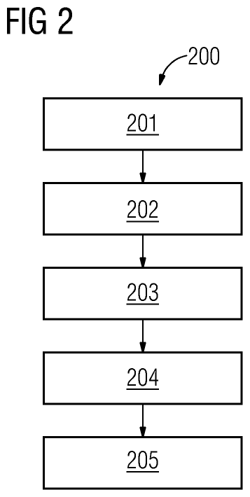

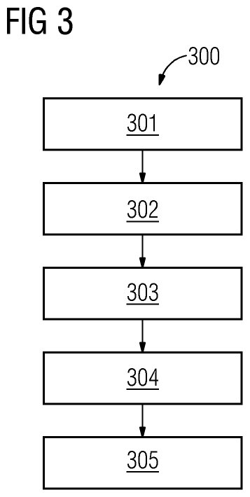

At step 104, the substrate bound to nucleic acids is washed using a wash buffer. The method steps for washing the substrate bound to nucleic acids is elaborated in further detail in FIG. 2. At step 105, the nucleic acids are eluted from the substrate using an elution buffer. The method steps for eluting the nucleic acids from the substrate is further elaborated in FIG. 3.

FIG. 2 illustrates one embodiment of a method 200 of washing the substrate bound to the nucleic acid. At step 201, the substrate bound to the nucleic acids is introduced to a wash buffer. In an embodiment, the substrate bound to the nucleic acids is aspirated into a pipette tip along with the wash buffer. The wash buffer enables removal of contaminants, salts, etc. from the sample or from the upstream buffers used in the method. In an embodiment, a plurality of wash buffers may be used for washing the substrate bound to the nucleic acids. In the present embodiment, three wash buffers are used for washing the substrate bound to nucleic acids. A first wash buffer may include a chaotrope with a concentration range of 1 to 10 M, a salt in the concentration range of 1 to 300 mM, ethanol in a concentration range of 20 to 50%, and preservatives. The pH of the first wash buffer may be in the range of 4 to 6. At step 202, a non-orbital movement is introduced in the pipette tip to provide that the wash buffer comes in complete contact with the substrate bound to nucleic acids. In an embodiment, the non-orbital movement may be introduced in the pipette tip using a magnet. For example, the magnet may be moved along the length of the pipette tip, with alternating magnetic fields on the opposite sides of the outer surface of the pipette tip, forcing the substrate bound to the nucleic acids to traverse through the length and breadth of the tip. The movement of the magnet also causes the substrate bound to the nucleic acids to move in a non-orbital way inside the pipette tip.

At step 203, a second wash buffer is introduced in the pipette tip and the step of washing the substrate bound to nucleic acids is repeated. The second wash buffer includes a salt in the concentration range of 1 to 20 mM, ethanol in the concentration range of 50 to 100%, and preservatives. The pH of the second wash buffer may be in the range of 4 to 6. In an embodiment, the step of washing the substrate bound to nucleic acids is repeated with a third wash buffer, at step 204. The third wash buffer includes a salt in the concentration range of 1 to 20 mM, a detergent in the concentration range of 0.05 to 2%, and preservatives. The pH of the third wash buffer may be in the range of 4 to 6. In an embodiment, an air gap may be introduced in the pipette tip while aspirating the third wash buffer. At step 205, an Eppendorf tube may be placed underneath the tip, and a magnet is introduced at the bottom of the Eppendorf tube. The substrate bound to nucleic acids is separated from the microtip, leaving the wash buffer in the pipette tip. The separated substrate bound to nucleic acids may then be processed downstream.

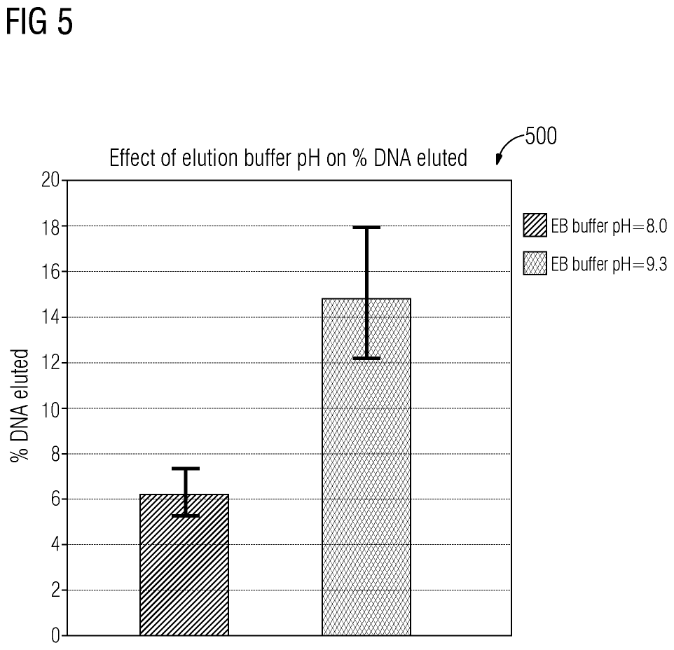

FIG. 3 illustrates a flowchart of one embodiment of a method 300 of eluting the nucleic acids from the substrate. At step 301, the washed substrate bound to nucleic acids is introduced to an elution buffer. Elution buffer enables separation of bound nucleic acid from the substrate. In an embodiment, the elution buffer has a high pH in the range of 8 to 9.5 and includes Tris HCl and EDTA. An elution buffer with a higher pH value enables efficient elution of nucleic acids. At step 302, the elution buffer is subjected to an increase in temperature, ranging between 70° C. and 95° C. Increasing the temperature of the elution buffer enhances the efficiency with which the nucleic acids are eluted by minimizing the wash buffer carry-over. Further, increasing pH of the elution buffer enables better elution of nucleic acids. As depicted in a graph 500 in FIG. 5, percentage of nucleic acids eluted increases with increase in pH of the elution buffer. In the embodiment, an elution buffer with a pH of 9.3 enables elution of approximately 15% of nucleic acids from the substrate in comparison to approximately 6% of nucleic acids elution with an elution buffer of pH 8.0.

In an embodiment, the mixture of elution buffer and the substrate bound to nucleic acids may be incubated and subjected to shaking for 0.5 to 3 minutes. At step 303, the eluate is aspirated into a new pipette microtip, where the eluate includes the substrate bound to the nucleic acids. The microtip is a narrow 20 μL tip. When the eluate is aspirated into the microtip, an air gap is introduced at the tip end of the microtip. An Eppendorf tube may be placed underneath the tip, and a magnet is introduced at the bottom of the Eppendorf tube. At step 304, the substrate is separated from the microtip, leaving the nucleic acids in the elution buffer in the microtip. This phenomenon is termed as ‘air jump’. At step 305, an elution wash may be performed on the substrate in the Eppendorf tube to provide that any remaining nucleic acid bound to the substrate is also eluted. The elution wash is performed by introducing the elution buffer to the substrate at room temperature and repeating the step of air jump to separate the eluate from the substrate. This provides maximum recovery of the nucleic acids from the substrate.

The embodiments described above enable detection of target nucleic acids faster and more efficiently.

TABLE 1
Comparison of nucleic acid yield for the present invention
v. Versant ® sample preparation method

| | | | | % DNA | |
|---|---|---|---|---|---|
| | | % DNA | % DNA | eluted | Total |
| Amount | Method | bound to | eluted (1^{st} | (Elution | yield of |
| of DNA | employed | substrate | elution) | wash) | DNA (%) |
| 100 ng | Standard | 99.83% ± | 42.77% ± | — | 42.77% ± |
| | Versant ® | 0.31% | 5.24% | | 5.24% |
| 100 ng | Present | 96.66% ± | 104.43% ± | 7.53% ± | 108.6% ± |
| | Invention | 1.48% | 14.11% | 4.16% | 12.8% |
| 1000 ng | Standard | 96.25% ± | 51.83% ± | — | 51.58% ± |
| | Versant ® | 1.07% | 3.3% | | 3.3% |
| 1000 ng | Present | 98.81% ± | 87.49% ± | 14.07% ± | 112.06% ± |
| | Invention | 0.38% | 34.27% | 5.31% | 16.49% |

The above table provides a comparison of nucleic acid yield from following the present embodiments in comparison with a Versant® sample preparation method. The method outlined in the present embodiments achieves nearly 100% recovery of nucleic acids from the sample. The present embodiments may be tailor made to many types of samples and targets with minimal upstream changes. Additionally, the present embodiments are amenable to automation and may be adapted to be used in a decentralized set-up with minimal user intervention. Yet another advantage of the present embodiments is that the method is very fast; for example, the extraction of nucleic acids from a virus spiked plasma sample is performed in 5 to 10 minutes. Similarly, the extraction of bacteria and fungi spiked whole blood sample is performed is 7 to 12 minutes. Further, the unique technique of separating the eluate from the substrate through the air gap in the microtip provides quick and efficient elution with minimal carry-over of wash buffer and maximum release of nucleic acid from the substrate.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials, and embodiments, the invention is not intended to be limited to the particulars disclosed herein; rather, the invention extends to all functionally equivalent structures, methods, and uses, such as are within the scope of the appended claims. Those skilled in the art, having the benefit of the teachings of this specification, may give effect to numerous modifications thereto, and changes may be made without departing from the scope and spirit of the invention in its aspects.

1. A method of extracting nucleic acids from a sample, the method comprising:
receiving the sample comprising nucleic acids to be extracted;
lysing cells in the sample, such that the nucleic acids are released;
introducing the released nucleic acids to a substrate, wherein the nucleic acids bind to the substrate;
washing the substrate bound to the nucleic acids; and
eluting the nucleic acids from the substrate;
wherein the introducing of the released nucleic acids to the substrate, the washing of the substrate bound to the nucleic acids, and the eluting of the nucleic acids employ non-orbital shaking of the substrate and the nucleic acids.

2. The method of claim 1, wherein the lysing comprises lysing the cells in the sample using ultrasound.

3. The method of claim 1, wherein the lysing comprises lysing the cells in the sample using a chemical lysis buffer, and
wherein the chemical lysis buffer comprises a chaotrope, a detergent, a buffering agent, or any combination thereof.

4. The method of claim 3, wherein the chemical lysis buffer is subjected to an increase in temperature after the chemical lysis buffer is brought in contact with the sample, and
wherein the increase in temperature is in a range of 60° C. to 90° C.

5. The method of claim 1, wherein washing the substrate bound to the nucleic acid comprises:
introducing the substrate bound to the nucleic acids to a wash buffer in a receptacle; and
subjecting the substrate bound to the nucleic acids to a non-orbital movement in the receptacle.

6. The method of claim 1, wherein the substrate is a silica coated paramagnetic bead.

7. The method of claim 5, wherein the non-orbital movement is introduced in the receptacle using one or more movable magnets.

8. The method of claim 5, wherein the non-orbital movement is introduced in the receptacle using a vortexer.

9. The method of claim 1, wherein eluting the nucleic acids from the substrate further comprises:
introducing the washed substrate bound to the nucleic acids to an elution buffer;
subjecting the elution buffer to an increase in temperature, wherein the increase in temperature is in a range of 75° C. to 95° C., and wherein the elution buffer has a pH of 8 to 9.5; and
separating an eluate from the elution buffer.

10. The method of claim 9, wherein separating the eluate from the elution buffer further comprises:
aspirating the eluate into a new pipette microtip, wherein the eluate comprises the substrate bound to the nucleic acids, wherein the new pipette microtip is a 20 μL tip, and wherein an air gap is introduced at a tip end of the new pipette microtip; and
separating the substrate from the new pipette microtip using a magnet, such that the nucleic acids are left in the new pipette microtip.

11. The method of claim 1, wherein the sample is sputum, plasma, urine, cerebrospinal fluid, or any combination thereof.
